(19) United States
(12) Patent Application Publication
(10) Pub. No.: US 2024/0325687 A1
(43) Pub. Date: Oct. 3, 2024

(54) FORCE-LIMITING ACCESS SYSTEM AND METHOD OF USE

(71) Applicant: CALYXO, INC., Pleasanton, CA (US)

(72) Inventors: Joseph CATANESE, III, Oakland, CA (US); Ling TONG, Fremont, CA (US); Matthew YUREK, San Diego, CA (US)

(21) Appl. No.: 18/740,388

(22) Filed: Jun. 11, 2024

Related U.S. Application Data
(63) Continuation of application No. PCT/US2022/054289, filed on Dec. 29, 2022.
(60) Provisional application No. 63/295,151, filed on Dec. 30, 2021.

(51) Int. Cl. A61M 25/00

(52) U.S. Cl. CPC A61M 25/0041; A61M 25/0043; A61M 25/0068; A61M 2205/33; A61M 2205/581; A61M 2205/582; A61M 2205/583

(57) ABSTRACT

A force-limiting access system is provided and includes a distal tip section configured to advance within a body lumen; a shaft coupled with the distal tip section; and a handle coupled with the shaft via an interface. The interface is configured to maintain a positional relationship between the handle and the shaft while the shaft experiences a force less than a threshold force and to allow the handle to move with respect to the shaft when the shaft experiences a force greater than the threshold force.

### TECHNICAL FIELD

This disclosure relates generally to the treatment of the urinary system, including the kidneys, ureters, bladder, and urethra. More specifically, but not by way of limitation, this disclosure relates to a system, device, and method for providing access to the urinary system for therapeutic or diagnostic purposes while reducing, minimizing, or eliminating the likelihood of trauma to the anatomy of the urinary system.

### BACKGROUND

Minimally invasive medical procedures, both therapeutic and diagnostic, often rely on gaining access to a part of the anatomy via a body lumen. Frequently, a guidewire is used to first establish a path along the lumen and to the anatomical target. Next, an access sheath is advanced along the guidewire to provide access for therapeutic and/or diagnostic tools. The access sheath must be larger than the tools so that the tools can easily advance through the sheath. However, large access sheaths can cause damage to the lumens into which they are advanced.

The ureter, which connects the kidneys to the bladder, is one type of body lumen that can be damaged by a large diameter access sheath. Recently, the forces exerted on the ureter during ureteral access sheath deployment have been quantified (Kaler, et al., Journal of endourology 33.9 (2019): 712-718.). In this study, the threshold force that result in ureteral injury in an animal model was quantified. Among their findings that significant ureteral injury can routinely be avoided if the applied force is less than 4.84 N.

In a similar study, forces related to ureteral access sheath were evaluated in human subjects (Tapiero, et al. The Journal of Urology (2021): 10-1097.). They concluded that ureteral access sheath associated ureteral injury can be averted by limiting the insertion force to less than or equal to 6 N.

One practical barrier to the routine implementation of these findings is that a force gauge is required to monitor the forces being exerted. Configuring a force gauge and monitoring may be impractical for many operative settings. An easy-to-use and scalable solution to limiting trauma that does not require a force gauge is needed to reduce ureteral trauma.

Thus, there is an unmet need for access devices and methods that reduce, minimize, or eliminate the likelihood of trauma to the anatomy of the urinary system.

### SUMMARY

In accordance with one embodiment, a force-limiting access system is provided. The system comprises a distal tip section configured to advance within a body lumen; a shaft coupled with the distal tip section; and a handle coupled with the shaft via an interface. The interface can be configured to maintain a positional relationship between the handle and the shaft while the shaft experiences a force less than a threshold force and to allow the handle to move with respect to the shaft when the shaft experiences a force greater than a threshold force.

In accordance with another embodiment, a force-limiting access system is provided, comprising a distal tip section configured to advance within a body lumen; a shaft coupled with the distal tip section; and a handle coupled with the shaft via an interface. The interface is configured to provide tactile feedback, audible feedback, visual feedback, or combinations thereof to a user when the shaft experiences a force greater than a threshold force.

In accordance with another embodiment, a kit for providing access to an anatomical target via a body lumen (e.g., urinary system, including kidney, ureters, bladder and/or urethra) is provided. The kit comprises a handle coupled with a proximal shaft via an interface, wherein the interface is configured to maintain the positional relationship between the handle and the proximal shaft while a force less than a threshold force is transmitted to the proximal shaft and to allow the handle to move with respect to the proximal shaft when a force greater than the threshold force is transmitted to the proximal shaft. The kit can include a set of removable shafts, each removable shaft configured to be coupled with the proximal shaft and configured to transmit force to the proximal shaft.

In accordance with another embodiment, a kit for providing access to an anatomical target via a body lumen (e.g., urinary system, including kidney, ureters, bladder and/or urethra) is provided, comprising a handle coupled with a proximal shaft via an interface, wherein the interface is configured to provide tactile feedback, audible feedback, visual feedback, or combinations thereof to a user when the shaft experiences a force greater than a threshold force. The kit can include a set of removable shafts, each removable shaft configured to be coupled with the proximal shaft and configured to transmit force to the proximal shaft.

In accordance with another embodiment, a method of accessing an anatomical target via a body lumen (e.g., urinary system, including kidney, ureters, bladder and/or urethra) is provided. The method comprises advancing a force-limiting access system with the body lumen toward an anatomical target, wherein the force-limiting access system comprises a handle coupled with a shaft via an interface configured to maintain a positional relationship between the handle and the shaft while a distal tip section coupled with the shaft experiences forces less than a threshold force and to allow the handle to move with respect to the shaft when the distal tip section experiences a force greater than the threshold force. The method can also include noting any feedback related to movement of the handle with respect to the shaft, where the feedback is tactile, audible, visual, or combinations thereof.

### BRIEF DESCRIPTION OF THE FIGURES

Features, aspects, and advantages of the present disclosure are better understood when the following Detailed Description is read with reference to the accompanying drawings.

FIG. 1 illustrates one view of a force-limiting access system according to certain aspects of the disclosure.

FIG. 2 illustrates one view of another force-limiting access system according to certain aspects of the disclosure.

FIG. 3 illustrates a side view of a distal end region of a force-limiting access system according to certain aspects of the disclosure.

FIG. 4 illustrates a side view of a proximal end region of a force-limiting access system according to certain aspects of the disclosure.

FIG. 5 illustrates a perspective view of a proximal end region of a force-limiting access system according to certain aspects of the disclosure.

FIG. 6A illustrates a cross-sectional side view of a proximal end region of a force-limiting access system according to certain aspects of the disclosure.

FIG. 6B illustrates a cross-sectional side view of a proximal end region of a force-limiting access system according to certain aspects of the disclosure.

FIG. 7 illustrates a perspective view of a proximal end region of a force-limiting access system according to certain aspects of the disclosure.

FIG. 8A illustrates a cross-sectional side view of a proximal end region of a force-limiting access system according to certain aspects of the disclosure.

FIG. 8B illustrates a cross-sectional side view of a proximal end region of a force-limiting access system according to certain aspects of the disclosure.

FIG. 9A illustrates a cross-sectional side view of a proximal end region of another force-limiting access system according to certain aspects of the disclosure.

FIG. 9B illustrates a cross-sectional side view of a proximal end region of another force-limiting access system according to certain aspects of the disclosure.

FIG. 10A illustrates a cross-sectional side view of a portion of a proximal end region of another force-limiting access system according to certain aspects of the disclosure.

FIG. 10B illustrates a cross-sectional side view of a portion of a proximal end region of another force-limiting access system according to certain aspects of the disclosure.

FIG. 11A illustrates a cross-sectional side view of a portion of a proximal end region of another force-limiting access system according to certain aspects of the disclosure.

FIG. 11B illustrates a cross-sectional side view of a portion of a proximal end region of another force-limiting access system according to certain aspects of the disclosure.

FIG. 12A illustrates a cross-sectional side view of a portion of a proximal end region of another force-limiting access system according to certain aspects of the disclosure.

FIG. 12B illustrates a cross-sectional side view of a portion of a proximal end region of another force-limiting access system according to certain aspects of the disclosure.

FIG. 13 illustrates a side view of a portion of a distal end region of another force-limiting access system according to certain aspects of the disclosure.

FIG. 14 illustrates a side view of a portion of a proximal end region of another force-limiting access system according to certain aspects of the disclosure.

FIG. 15 illustrates a cross-sectional end view of a portion of a middle region of another force-limiting access system according to certain aspects of the disclosure.

FIG. 16 illustrates a method of using the force-limiting access system according to certain aspects of the disclosure.

### DETAILED DESCRIPTION

It is understood that this disclosure, in many respects, is only illustrative of the various alternative device examples of the present invention. Changes may be made in the details, particularly in matters of shape, size, material, and arrangement of various device components without exceeding the scope of the various examples in the disclosure.

When a feature or element is herein referred to as being “connected,” “attached,” or “coupled” to another feature or element, it can be directly connected, attached, or coupled to the other feature or element or intervening features or elements may be present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing examples only and is not intended to be limiting of the invention. For example, as used herein, the singular forms “a”, “an,” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any combinations of one or more of the associated listed items.

The spatially relative terms, “proximal,” “distal,” and the like, may be used herein for ease of description to describe one element's or feature's relationship to another. It will be understood that proximal describes a spatial location closer to the user or the intended position of the user while distal describe a location farther from the user or the intended position of the user. Further, when used with respect to a minimally invasive device like a catheter, proximal and distal locations refer to the portion of the device that is intended to be closer to or farther from the user, respectively, and do not change when the device is in use.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions.

As used herein in the specification and claims, the term “handle” means a proximal member of the system described here that is configured to be gripped and/or manipulated by the hands of a user. The handle can include functionality other than the ability to be gripped or manipulated, and can include, for example, hubs with one or more ports or other features and functionality consistent with common usage in minimally invasive procedures.

A force-limiting access system can include a handle of an access sheath or dilator or other similar device that can provide feedback to a user where the feedback corresponds to a pre-determined force being experienced by a distal section of the access sheath or dilator or other similar device. The feedback can be tactile, audible, visual, or combinations thereof. The handle slips with respect to a shaft to prevent application of excess force to the anatomy and to provide feedback to the user.

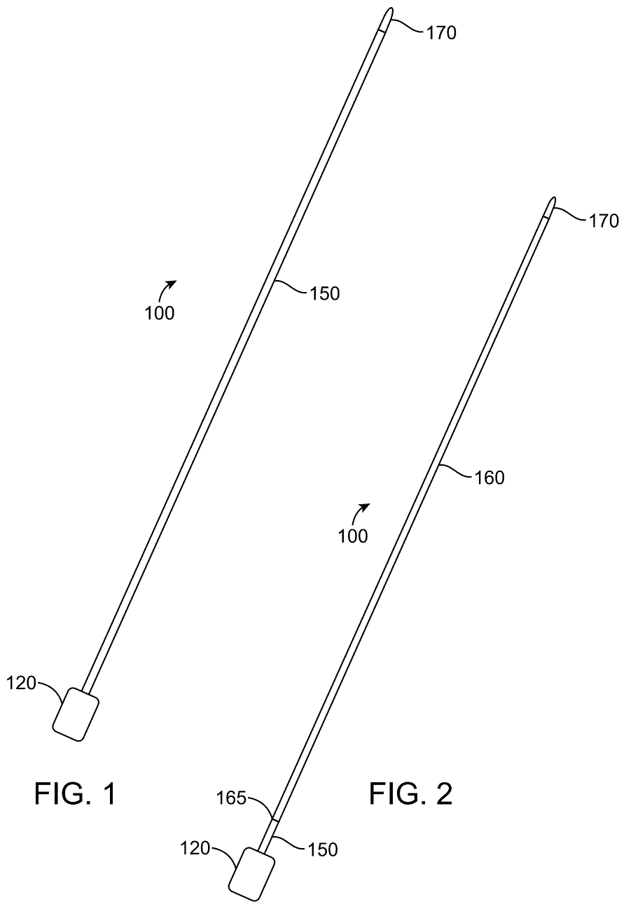

FIG. 1 illustrates one view of a force-limiting access system according to certain aspects of the disclosure. The system 100 includes a handle 120 coupled with a shaft 150 and the shaft 150 includes a distal tip section 170. The shaft 150 extends from its coupling with the handle 120 to the distal tip section and forms the entire elongate portion of the system that connects the handle 120 to the distal tip section 170. The shaft 150 is configured to navigate a body lumen of a patient and is constructed using conventional techniques to provide the mechanical properties commonly associated with the shafts of similar devices, such as guiding catheters, catheter introducers/dilators, therapeutic catheters, and/or diagnostic catheters. That is, the shaft 150 exhibits sufficient mechanical properties including, but not limited to, column strength, flexibility, torque response, and lubricity. The distal tip section 170 is configured to provide atraumatic interactions with tissue as the shaft 150 navigates the body lumen and is constructed using conventional techniques to provide the mechanical properties commonly associated with the distal tips of similar devices. That is, the distal tip exhibits sufficient mechanical properties including, but not limited to, flexibility, softness, and lubricity. Generally, the distal tip section 170 will be more flexible and softer than the shaft 150. Alternatively, the distal tip section 170 may be similar in stiffness to the shaft 150 and a separate introducer device may be used in conjunction with the access device. This separate introducer device could have a tip that extends distally past the shaft 150 and is more flexible and softer than the shaft 150. The separate introducer could also have a tapered lead-in with an inner diameter sized and configured to accommodate a guidewire, and an maximum outer diameter to provide a smooth transition to the shaft 150.

FIG. 2 illustrates one view of another force-limiting access system according to certain aspects of the disclosure. The system 100 includes a handle 120 coupled with a shaft 150 at proximal region of the shaft 150. A distal region of the shaft 150 is coupled with a removable shaft 160 via a coupling 165. The removable shaft 160 includes a distal tip section 170. The main difference between the system of FIG. 1 and the system of FIG. 2 is the removable shaft 160.

The system 100 of FIG. 2 allows for multiple different removable shafts 160 to be coupled with the shaft 150 and the system 100 can be provided in a kit that includes a handle 120 connected to a shaft 150 and several removable shafts 160 each having a distal tip section 170. The removable shafts 160 provided in the kit can have different properties and/or dimensions. For example, a set of removable shafts 160 in the kit can be provided in different sizes in the French scale of catheter sizes. The set of removable shafts 160 in the kit could be provided in 8 Fr, 9 Fr, 10 Fr, 11 Fr, and 12 Fr, for example. In these examples, each of the removable shafts 160 is configured to be coupled to the shaft 150 via the coupling 165. In some examples, the entire shaft 150 and the coupling 165 is positioned within the handle 120 rather than distal to the handle 120 as illustrated in FIG. 2. In such examples, the principles of operation of the system remain the same as described herein.

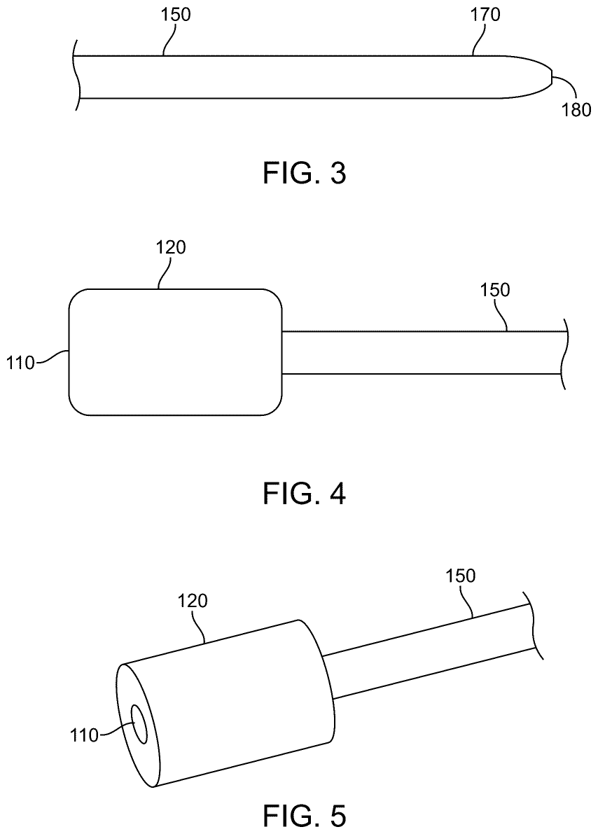

FIG. 3 illustrates a side view of a distal end region of a force-limiting access system according to certain aspects of the disclosure. The distal tip section 170 includes a distal opening 180 that is the distal end of a lumen within the distal tip section 170 and the shaft 150. This lumen is defined by the tubular configuration of both the distal tip section 170 and the shaft 150. The lumen can be used to advance the system over a guidewire that has been previously placed in a body lumen. While FIG. 3 illustrates a system in which the shaft 150 runs distally to the distal tip section 170, the system of FIG. 2 is also within the scope of this aspect of the disclosure. That is, a removable shaft 160 is configured as a tubular structure to include a lumen that can be used to advance systems using removable shafts 160 over a guidewire.

FIG. 4 illustrates a side view of a proximal end region of a force-limiting access system and FIG. 5 illustrates a perspective view of a proximal end region of a force-limiting access system according to certain aspects of the disclosure. The handle 120 is coupled with the shaft 150 and the handle 120 includes a proximal opening 110, which provides access to the lumen within the shaft 150. When the system is advanced over a guidewire, the guidewire enters the lumens of the system through the distal opening 180 and exist the lumens of the system through proximal opening 110. The system of FIG. 2 is also within the scope of this aspect of the disclosure. That is, a removable shaft 160 configured to include a lumen is connected with the lumens of the shaft 150 and the handle 120 such that the system can be advanced over a guidewire.

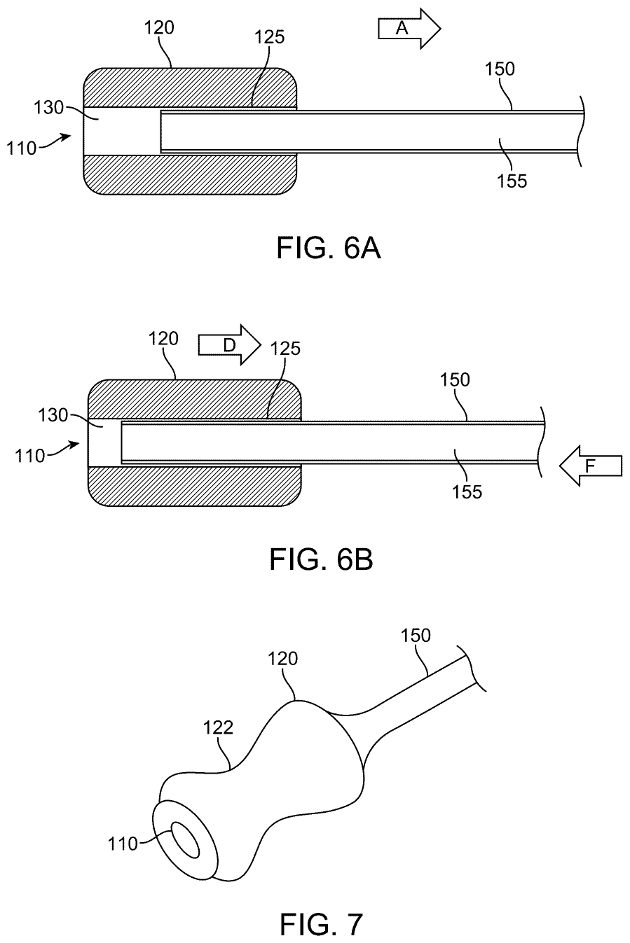

FIG. 6A and FIG. 6B each illustrate a cross-sectional side view of a proximal end region of a force-limiting access system according to certain aspects of the disclosure. The structure of the shaft 150 defines a shaft lumen 155 that is in fluid communication with a handle lumen 130. The shaft 150 is configured such that there is an interface 125 between the outer structure of the shaft 150 and the inner structure of the handle lumen 130 of the handle 120.

As described in further detail herein, the interface 125 can take various forms and each of these forms operates on the principle of providing a threshold force to maintain the positional relationship between the handle 120 and the shaft 150. That is, while the shaft 150 experiences forces less than a threshold amount, the handle 120 and the shaft 150 will move in concert such that moving the handle 120 by a first amount also moves the shaft 150 by that first amount. When the shaft 150 experiences forces at or above the threshold amount, the static force of the interface 125 is overcome and the handle 120 will move with respect to the shaft 150.

FIG. 6A and FIG. 6B together illustrate an example of the shaft 150 experiencing a force that overcomes the static force of the interface 125 and the resulting movement of the handle 120 with respect to the shaft 150. In this example, the system is being advanced within the body lumen of a patient in the direction of arrow A shown in FIG. 6A.

Initially, the system encounters minimal opposition from the body lumen to being advanced in the direction of arrow A. At one point in time as the system is advanced in the body lumen, the system experiences forces that oppose movement in the direction of arrow A. These forces are due to the inner diameter of the body lumen being just larger than the outer diameter of the widest portion of the distal tip of the system. The forces are communicated along the shaft 150 (or along the removable shaft 160 and to the shaft 150). But at this point in time, the forces are below the threshold static force of the interface 125 and the handle 120 and shaft 150 are still able to move together the same amount.

FIG. 6B illustrates a point where the system has encountered forces represented by arrow F and above the threshold static force of the interface 125. As a result, the handle 120 “breaks free” from being fixedly coupled to the shaft 150 and is free to move in the direction of arrow D. Movement of the handle 120 with respect to the shaft 150 provides tactile feedback to the user that the threshold force of the interface 125 has been exceeded. The user therefore knows that the distal tip section 170 has encounter a part of the body lumen that is too narrow to safely advance the system. The movement of the handle 120 with respect to the shaft 150 can also trigger audible and/or visual signals in addition to tactile feedback.

In some aspects of the disclosure, the system is configured such that shaft 150 extends entirely through the handle 120. In such aspects, the initial position of the handle 120 with respect to the shaft 150 is such that there is a portion of the shaft 150 (and the shaft lumen 155) proximal of the proximal side of the handle 120. That is, the proximal opening of the shaft lumen 155 need not be within the handle 120 in an initial configuration but rather the proximal opening of the shaft lumen 155 can be proximal of the handle 120 in an initial configuration.

In some aspects of the disclosure, the shaft 150 (or the removable shaft 160) includes a shaft handle portion fixed to the shaft for use in navigating the system through portions of the anatomy where the force-limiting features are not needed or are undesirable. For example, when navigating through the urethra and bladder to the target anatomy of the kidney, the force-limiting features may not be needed, and the user can navigate the system using the shaft handle portion. When the distal tip section reaches the opening of the ureter in the bladder, the user can then begin to navigate the system using the force-limiting features of the handle 120. Alternatively, the handle 120 can include a lock-out feature that fixes the handle 120 to the shaft 150. In this alternative aspect, the lock-out feature maintains the fixed positional relationship between the handle 120 and the shaft 150 and prevent the force-limiting features from activating. Like the shaft handle portion, the lock-out feature allows the user to navigate the system in situations where the force-limiting features may not be needed. Lock-out features may be in the form of pins, recesses, screws, threads, or any other mechanical, electrical, magnetic, electromagnetic, and/or electro-mechanical feature that is configured to fix the handle to the shaft and/or prevent activation of the force-limiting features of the system during navigation.

FIG. 7 illustrates a perspective view of a proximal end region of another force-limiting access system according to certain aspects of the disclosure. A handle 120 in coupled with a shaft 150 and includes a proximal opening 110, which provides access to a lumen within the shaft 150. The handle 120 includes a gripping region 122, which is configured to be easily gripped by a user. The gripping region 122 allows the user to manipulate the handle 120 and can be configured to transmit tactile feedback to the user.

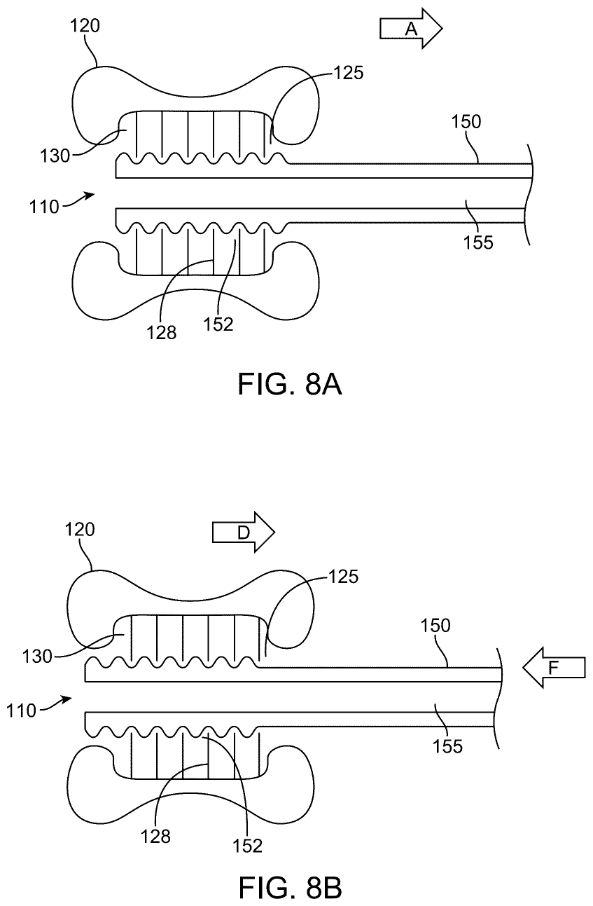

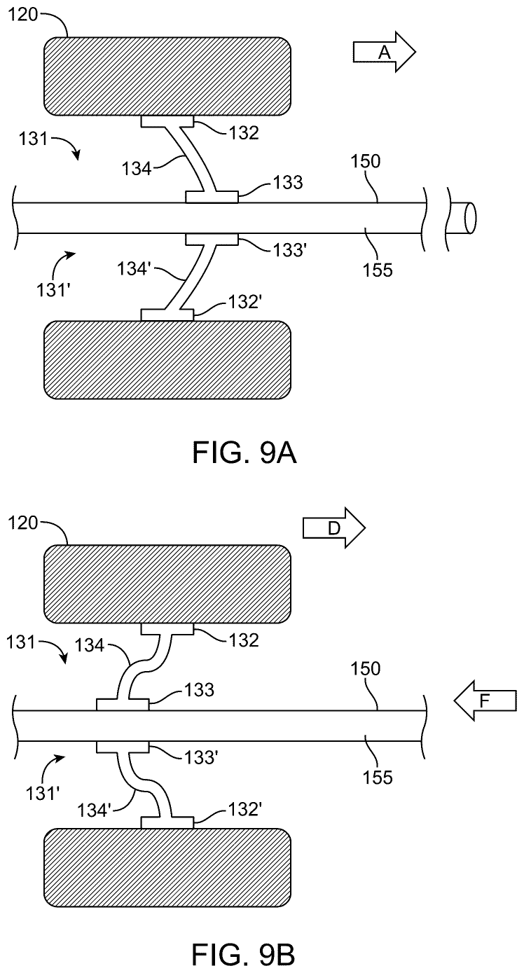

FIG. 8A and FIG. 8B each illustrate a cross-sectional side view of a proximal end region of another force-limiting access system according to certain aspects of the disclosure. Like FIG. 6A and FIG. 6B, FIG. 8A and FIG. 8B together illustrate an example of the shaft 150 experiencing a force that overcomes the static force of the interface 125 and the resulting movement of the handle 120 with respect to the shaft 150. In this example, the system is being advanced within the body lumen of a patient in the direction of arrow A shown in FIG. 8A. The shaft 150 includes a shaft lumen 155 that is disposed with a handle lumen 130 such that there is an interface 125 between the outer structure of the shaft 150 and the inner structure of the handle lumen 130 of the handle 120.

FIG. 8A and FIG. 8B illustrate one example of an interface 125 that includes features that provide feedback to a user that a threshold force has been exceeded. There are other examples of mechanical, electrical, magnetic, electromagnetic, and/or electro-mechanical features that will function to provide the force-limiting characteristics of the interface disclosed herein.

The interface 125 of FIG. 8A and FIG. 8B includes a set of features on the shaft 150 and the handle 120 that interact with each other to provide the force-limiting characteristics of the interface 125. The handle 120 includes a set of projections 128 that corresponds to a series of detents 152 included in the shaft 150. The projections 128 have a flexibility that when combined with the height of the detents 152 results in the interface 125 maintaining a static positional relationship between the handle 120 and the shaft 150 when the force on the shaft 150 is below a threshold amount. Like in FIG. 6A and FIG. 6B, FIG. 8A and FIG. 8B illustrate that the system can experience forces that oppose movement in the direction of arrow A. Such forces are represented by arrow F. FIG. 8B illustrates that the handle 120 moves in a direction represented by arrow D because the set of projections 128 deflect distally by an amount sufficient to overcome the height of the set of detents 152. Advantageously, the presence of a set of projections 128 and detents 152 allows for the system to encounter multiple instances of forces exceeding a threshold force. That is, the handle 120 “breaks free” from the shaft 150 and slides in the direction of the arrow D but is still configured to move in concert with the shaft 150 if the forces encountered by the system do not exceed the threshold force because several of the set of projection 128 are aligned with several of the set of detents 152. In this example of a set of projections 128 and detents 152 it may be necessary to progressively vary the flexibility of the projections 128 and/or the height of the detents 152. That is, when fewer projections 128 are interacting with detents 152, that interaction must still be configured to “break free” at the same threshold force.

In some aspects of the disclosure, the set of features on the shaft 150 (such as the set of detents 152) are more numerous than the set of corresponding features on the handle 120. In such aspects, the handle 120 and shaft 150 interact to provide a comparatively longer range of motion for the force-limiting features than the aspect of the disclosure in which the features on the shaft extend only within the handle.

Further, the set of features on the shaft can be configured to provide consistent or variable threshold static forces. That is, one section of the set of features can be tuned to provide a first threshold force and another section of the features can be tuned to provide a second threshold force. Thus, a single shaft can interact with a handle to provide different threshold forces, where the threshold force is determined by the position of the handle with respect of the set of features on the shaft.

More generally, the force-limiting access systems disclosed herein can included interfaces that are tuned to “break free” at various, different threshold forces. A kit can include a set of handles and a set of shafts and/or dilators that are tuned to different force thresholds.

FIG. 9A and FIG. 9B each illustrate a cross-sectional side view of a proximal end region of another force-limiting access system according to certain aspects of the disclosure. Like FIG. 6A and FIG. 6B, FIG. 9A and FIG. 9B together illustrate an example of the shaft 150 experiencing a force that overcomes the static force of an interface and the resulting movement of the handle with respect to the shaft 150. In this example, the system is being advanced within the body lumen of a patient in the direction of arrow A shown in FIG. 9A. The shaft 150 includes a shaft lumen 155 and is disposed within a handle 120 such that there is an interface feature 131, 131′ between the outer structure of the shaft 150 and the inner structure of the handle 120.

The interface feature 131, 131′ couple the shaft 150 with the handle 120 to provide force-limiting characteristics. The interface feature 131, 131′ include a handle coupling 132, 132′ and a shaft coupling 133, 133′. These couplings attach the handle 120 to the shaft 150 and allow the bistable element 134, 134′ to function as the force-limiting structure in this example.

The bistable element 134, 134′ is defined by the characteristic that there are two structural configurations in which it is stable. FIG. 9A illustrates a first stable configuration in which the bistable element 134, 134′ is generally linear and FIG. 9B illustrates a second stable configuration in which the bistable element 134, 134′ is generally curved. The bistable element 134, 134′ is designed to maintain one of the two stable configurations and the bistable element 134, 134′ transitions between the two stable configurations only when the interface feature 131, 131′ experiences a force above a threshold amount.

Like in FIG. 6A and FIG. 6B, FIG. 9A and FIG. 9B illustrate that the system can experience forces that oppose movement in the direction of arrow A. Such forces are represented by arrow F. FIG. 9B illustrates that the handle 120 moves in a direction with represented by arrow D because the threshold amount of force has been exceeded, causing the bistable element 134, 134′ to transition from the first stable configuration to the second stable configuration. That is, the handle 120 moves with respect to the shaft 150 in the direction D because the force F is greater than the threshold amount. In this way, the interface feature 131, 131′ acts as a force-limiting structure.

FIG. 9A and FIG. 9B illustrate that the upper and lower portions of the handle 120 are coupled to the interface feature 131, 131′ via the handle coupling 132, 132′. Preferably, this is a fixed coupling such that the handle 120 and the handle coupling 132, 132′ move together as if they were a single unit. Similarly, the upper and lower surfaces of the shaft 150 are coupled to the interface feature 131, 131′ via the shaft coupling 133, 133′. Preferably, this is a fixed, but detachable, coupling such that the handle 120 and the shaft coupling 133, 133′ move together as if they were a single unit. In some cases, the shaft coupling 133, 133′ is detachable from the shaft 150 such that it can be repositioned on another portion of the shaft 150. Repositioning the shaft coupling 133, 133′ also repositions the entire handle 120 with respect to the shaft 150 in the examples in which the interface feature 131, 131′ is fixed to the handle. Detachable coupling between the shaft 150 and the shaft coupling 133, 133′ can be accomplished by a variety of attachment methods, including, but not limited to, adhesives (including pressure sensitive adhesives). Further, the handle 120 is repositionable on both the shaft 150 and the removable shaft 160 disclosed herein.

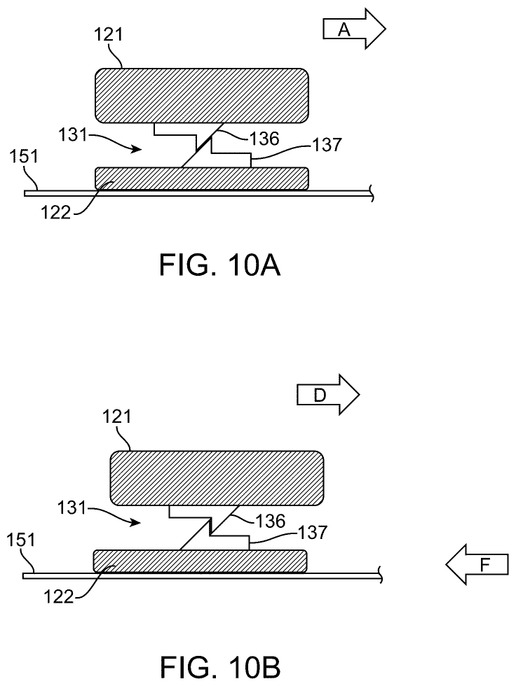

FIG. 10A and FIG. 10B illustrate cross-sectional side views of a portion of a proximal end region of another force-limiting access system according to certain aspects of the disclosure. In these views, an upper handle portion 121 and a lower handle portion 122 are illustrated and include an interface feature 131 between them. Like the example illustrated in FIG. 9A and FIG. 9B, the lower handle portion 122 can be detachably coupled with the shaft, which in FIGS. 10A and 10B is represented by shaft surface 151, or with a removable shaft.

The interface feature 131 of FIG. 10A and FIG. 10B includes a set of interlocking features 136, 137 that are configured to perform similarly to the bistable elements and the interfaces disclosed herein. That is, the set of interlocking features 136, 137 operate to provide force-limiting characteristics to enable the handle to advance the shaft until the shaft experiences force in excess of a threshold, at which point the handle “breaks-free” from the shaft and is free to move in the direction of arrow D.

The set of interlocking features 136, 137 are illustrated in FIG. 10A in contact with one another and capable of advancing the shaft (via shaft surface 151) in the direction of arrow A. The set of interlocking features 136, 137 are mechanically linked via interacting surfaces that have sufficient frictional force between them to maintain positional stability until a threshold force has been met. The frictional force on the surfaces of the set of interlocking features 136, 137 is accomplished using conventional methods, including, but not limited to, roughness, adhesiveness, micro-interlocking features, and the like. The set of interlocking features 136, 137 are also characterized by have sufficient flexibility and/or deformability such that the interlocking feature 136 can move distally with respect to the interlocking feature 137 in a similar manner to how the projections 128 flex.

FIG. 10A and FIG. 10B illustrate one set of interlocking features 136, 137, but some examples may include a plurality of sets of interlocking features 136, 137. Such an example can perform like the example illustrated in FIG. 8A and FIG. 8B in which the handle can continue to be used to advance the shaft even after having “broken free” one or more times.

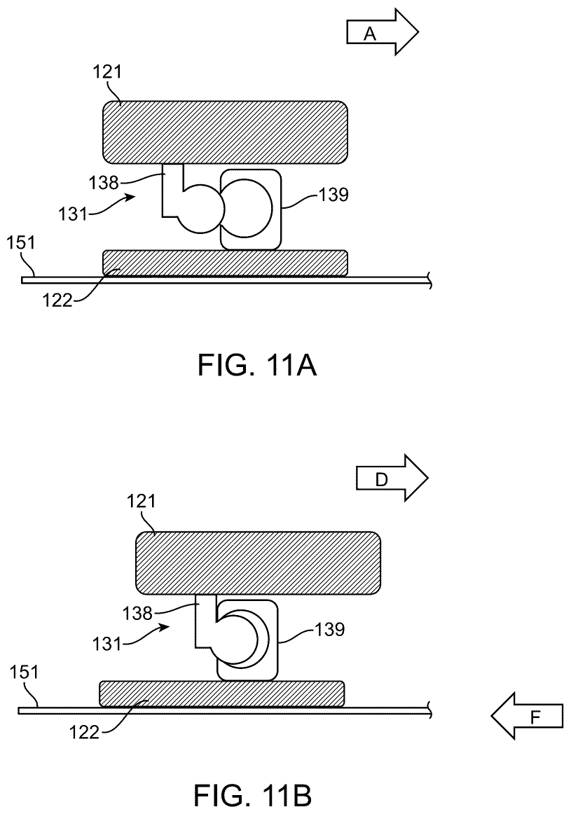

FIG. 11A and FIG. 11B illustrate cross-sectional side views of a portion of a proximal end region of another force-limiting access system according to certain aspects of the disclosure. In these views, an upper handle portion 121 and a lower handle portion 122 are illustrated and include an interface feature 131 between them. Like the example illustrated in FIG. 10A and FIG. 10B, the lower handle portion 122 can be detachably coupled with the shaft, which is represented by shaft surface 151, or with a removable shaft.

The interface feature 131 of FIG. 11A and FIG. 11B includes a set of interlocking features 138, 139 that are configured to perform similarly to the bistable elements and the interfaces disclosed herein. That is, the set of interlocking features 138, 139 operate to provide force-limiting characteristics to enable the handle to advance the shaft until the shaft experiences force in excess of a threshold, at which point the handle “breaks-free” from the shaft and is free to move in the direction of arrow D.

The set of interlocking features 138, 139 are illustrated in FIG. 11A in contact with one another and capable of advancing the shaft (via shaft surface 151) in the direction of arrow A. The interlocking feature 138 is substantially rigid while the interlocking feature 139 is comparatively flexible at least at its proximal side. The threshold force for the set of interlocking features 138, 139 is the force required to flex the proximal portion of the interlocking feature 139 such that the interface feature 131 takes on the configuration depicted in FIG. 11B. That is, FIG. 11A depicts a first stable configuration and FIG. 11B depicts a second stable configuration. In each stable configuration, the handle maintains positional stability with respect to the shaft, at least in the case of advancing the handle and shaft in the direction of arrow A.

In some examples of the interface features disclosed herein, the interface feature is a bistable element that can transition from the first stable configuration to the second stable configuration and back again to the first stable configuration. In other examples, the interface feature is a bistable element that is capable only of a transition from the first stable configuration to the second stable configuration while in use and must be reset to the first stable configuration through a process separate from simply moving the handle in the direction opposite of arrow D with sufficient force. The interface feature 131 in FIG. 11B is an example of one that can have a configuration in which it does not transition back to the first stable configuration from simply moving the handle in the direction opposite of arrow D with sufficient force but instead must be removed from the shaft and reset.

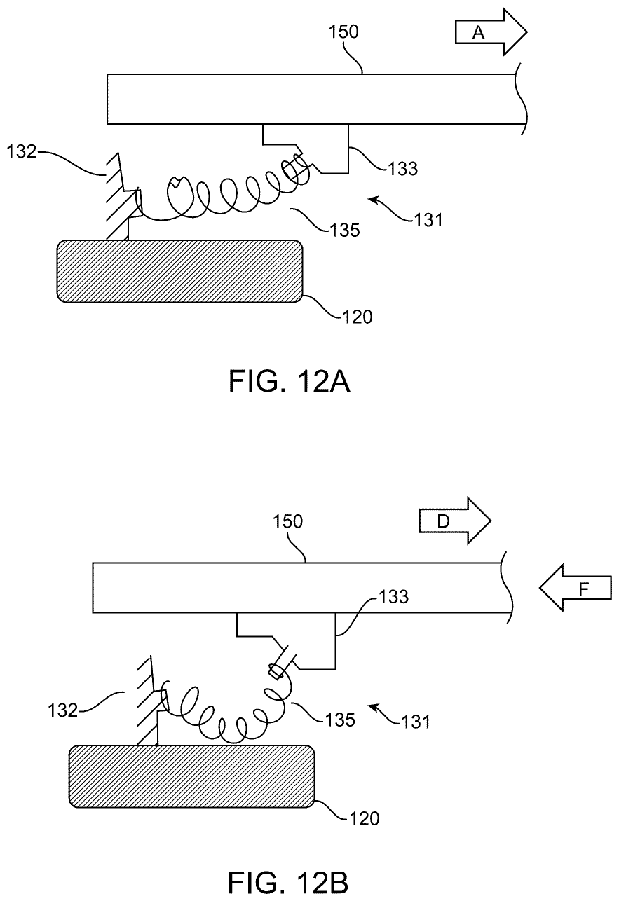

FIG. 12A and FIG. 12B illustrate a cross-sectional side view of a portion of a proximal end region of another force-limiting access system according to certain aspects of the disclosure. In FIG. 12A and FIG. 12B, the shaft 150 is coupled with a handle 120, although only the lower portion of handle 120 is illustrated. An interface feature 131 is coupled to both the shaft 150 and the handle 120 and includes a handle coupling 132 and a shaft coupling 133. A buckling member 135 is coupled with the handle coupling 132 and the shaft coupling 133 and is characterized by stiffness that resists buckling until the shaft 150 encounters a force in excess of a threshold force. Thus, the handle 120 moves with positional stability with respect to the shaft 150 as both are advanced in the direction of arrow A. When the shaft 150 encounters a force in the direction of arrow F, the buckling member 135 buckles and allows the handle 120 to move in the direction of arrow D with respect to the shaft 150. The buckling of buckling element 135 can create audible feedback, such as in the form of a click sound and/or tactile feedback, such as in the form of a slight vibration. The interface feature 131 of FIG. 12A and FIG. 12B can be advantageously combined with any of the other interface features disclosed herein to provide audible and/or tactile feedback or it can be the only source of force-limiting structure in the system.

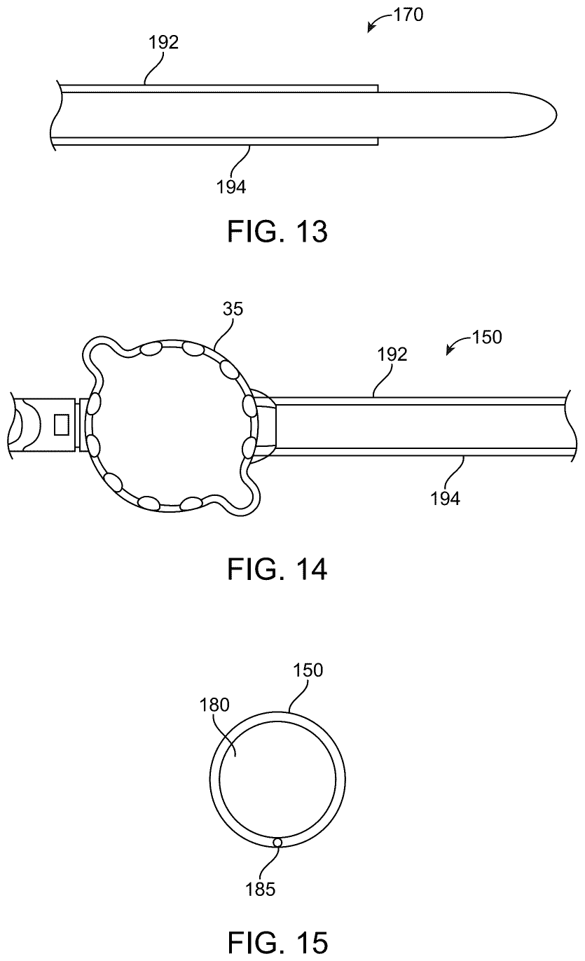

FIG. 13 illustrates a side view of a portion of a distal end region of another force-limiting access system according to certain aspects of the disclosure. The distal tip section 170 of the shaft includes one or more steering wires 192, 194. The steering wires 192, 194 are fixed to the distal tip section 170 and facilitate steering of the distal tip section 170 to help reduce the forces that may act on the shaft as the system is advanced through tissue or body lumens. FIG. 14 illustrates a side view of a portion of a proximal end region of another force-limiting access system according to certain aspects of the disclosure. The shaft 150 includes one or more steering wires 192, 194 fixed to the distal tip section 170 as illustrated in FIG. 13 and terminating at a steering mechanism 35. In some examples, it is preferable that the steering mechanism 35 and one or more steering wires 192, 194 are characterized by a return to a centered configuration when the steering mechanism is released by a user.

FIG. 15 illustrates a cross-sectional end view of a portion of a middle region of another force-limiting access system according to certain aspects of the disclosure. The shaft 150 includes one or more ports 182 at or near the distal end of the shaft. The port 182 is capable of delivering a chemical or biological agent that will relax tissue to reduce the forces encountered by advancing the system through tissue. The port 182 can be connected to the proximal end of the system via a lumen and the agent can be fluidly conducted through the lumen from the proximal end to the distal end. In some examples, the port 182 can be pre-loaded with an agent that elutes during use. In this example, the port 182 is similar to a reservoir and does not need to be in fluid communication with the proximal end of the system. In yet another example, the distal end of the shaft can include an inflatable member to mechanically dilate tissue to open a body lumen slightly prior to advancing the system, thereby reducing the forces encountered by advancing the system through tissue.

Any of the examples disclosed herein can further include a strain gauge to provide quantitative feedback to the user about the forces experienced by the system as it is advanced. The strain gauge can be mechanical, electrical, electromechanical, or of other suitable construction.

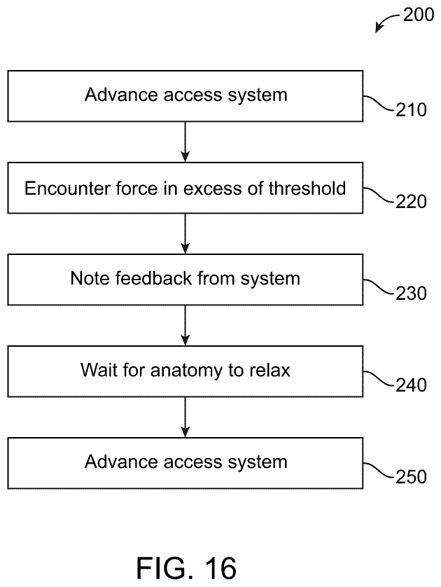

FIG. 16 illustrates a method of using the force-limiting access system according to certain aspects of the disclosure. In the method 200, a first step 210 includes advancing the force-limiting access system within a body lumen toward a target anatomy. In a second step 220, the access system encounters a force in excess of the threshold force to which the system has been tuned. In a third step 230, the user notes the feedback related to the second step, where the feedback is tactile, audible, visual, or combinations thereof. Because of such the feedback, which may include the tactile feedback of the system handle moving with respect to the system shaft, the fourth step 240 is that the user waits for the anatomy to relax. In some instances, the body lumen may be able to accommodate further advancement of the access system. After waiting, the user undertakes the fifth step 250 of attempting to advance the access system again. In some instances, the system will be able to be advanced. In other instances, the system will again experience forces in excess of the threshold force and the system handle will move with respect to the system shaft. In such instances, the user will retract the system from the body lumen and repeat the method with a smaller size shaft. For example, if the body lumen cannot accommodate a 12 Fr shaft, then the user can employ an 11 Fr shaft. In some instances, the method can include introducing an additional device or substant to decrease the resistance provided by the body lumen and/or to relax the anatomy. That is, the fifth step 250 may include the passive step of waiting with an active step of introducing a dilating device or therapeutic agent. Exemplary dilating devices include, but are not limited to, expandable balloon catheters and other mechanical dilators. Therapeutic agents include, but are not limited to, lubricants, vasodilators, and tissue relaxants.

In an aspect of the disclosure, a force-limiting access system includes a distal tip section configured to advance within a body lumen, a shaft coupled with the distal tip section, and a handle coupled with the shaft via an interface, wherein the interface is configured to maintain a positional relationship between the handle and the shaft while the shaft experiences forces less than a threshold force and to allow the handle to move with respect to the shaft when the shaft experiences forces greater than a threshold force.

In this or in combination with any other aspects of the disclosure, the interface comprises a mechanical, electrical, magnetic, electromagnetic, and/or electro-mechanical feature that allows the handle to move with respect to the shaft when the shaft experiences forces greater than a threshold force.

In this or in combination with any other aspects of the disclosure, the interface comprises a feature on the handle that allows the handle to move with respect to the shaft when the shaft experiences forces greater than a threshold force.

In this or in combination with any other aspects of the disclosure, the interface comprises a set of features on the handle that allows the handle to move with respect to the shaft when the shaft experiences forces greater than a threshold force.

In this or in combination with any other aspects of the disclosure, the interface comprises a feature on the shaft that allows the handle to move with respect to the shaft when the shaft experiences forces greater than a threshold force.

In this or in combination with any other aspects of the disclosure, the interface comprises a feature on the shaft that allows the handle to move with respect to the shaft when the shaft experiences forces greater than a threshold force.

In this or in combination with any other aspects of the disclosure, the interface comprises a set of features on the shaft that allows the handle to move with respect to the shaft when the shaft experiences forces greater than a threshold force.

In this or in combination with any other aspects of the disclosure, the interface comprises a feature or set of features that provides tactile feedback, audible feedback, visual feedback, or combinations thereof to a user when the shaft experiences forces greater than a threshold force.

In this or in combination with any other aspects of the disclosure, the interface comprises a projection on the handle and a detent on the shaft.

In this or in combination with any other aspects of the disclosure, the interface comprises a set of projections on the handle and a set of detents on the shaft.

In this or in combination with any other aspects of the disclosure, the interface is tuned to a pre-determined threshold force.

In this or in combination with any other aspects of the disclosure, the distal tip section is configured to transmit force to the shaft and at least a portion of the forces experienced by the shaft are transmitted to the shaft by the distal tip section.

In an aspect of the disclosure, a force-limiting access system includes a distal tip section configured to advance within a body lumen, a shaft coupled with the distal tip section, and a handle coupled with the shaft via an interface, wherein the interface is configured to provide tactile feedback, audible feedback, visual feedback, or combinations thereof to a user when the shaft experiences forces greater than a threshold force.

In this or in combination with any other aspects of the disclosure, the interface comprises a mechanical, electrical, magnetic, electromagnetic, and/or electro-mechanical feature that provides tactile feedback, audible feedback, visual feedback, or combinations thereof to a user when the shaft experiences forces greater than a threshold force.

In this or in combination with any other aspects of the disclosure, the interface comprises a feature on the handle that is configured to prevent excess force on the body lumen when the shaft experiences forces greater than a threshold force.

In this or in combination with any other aspects of the disclosure, the interface comprises a set of features on the handle that are configured to prevent excess force on the body lumen when the shaft experiences forces greater than a threshold force.

In this or in combination with any other aspects of the disclosure, the interface comprises a feature on the shaft that is configured to prevent excess force on the body lumen when the shaft experiences forces greater than a threshold force.

In this or in combination with any other aspects of the disclosure, the interface comprises a set of features on the shaft that are configured to prevent excess force on the body lumen when the shaft experiences forces greater than a threshold force.

In this or in combination with any other aspects of the disclosure, the distal tip section is configured to transmit force to the shaft and at least a portion of the forces experienced by the shaft are transmitted to the shaft by the distal tip section.

In an aspect of the disclosure, a kit for providing access to an anatomical target via a body lumen includes a handle coupled with a proximal shaft via an interface, wherein the interface is configured to maintain the positional relationship between the handle and the proximal shaft while the forces less than a threshold force are transmitted to the shaft and to allow the handle to move with respect to the proximal shaft when forces greater than a threshold force are transmitted to the shaft, and a set of removable shafts, each removable shaft configured to be coupled with the proximal shaft and configured to transmit force to the proximal shaft.

In this or in combination with any other aspects of the disclosure, the set includes removable shafts of different sizes.

In this or in combination with any other aspects of the disclosure, the set includes removable shafts of different French scale sizes.

In this or in combination with any other aspects of the disclosure, the interface comprises a mechanical, electrical, magnetic, electromagnetic, and/or electro-mechanical feature that allows the handle to move with respect to the proximal shaft when the removable shaft experiences forces greater than a threshold force.

In this or in combination with any other aspects of the disclosure, the interface comprises a feature on the handle that allows the handle to move with respect to the proximal shaft when the removable shaft experiences forces greater than a threshold force.

In this or in combination with any other aspects of the disclosure, the interface comprises a set of features on the handle that allows the handle to move with respect to the proximal shaft when the removable shaft experiences forces greater than a threshold force.

In this or in combination with any other aspects of the disclosure, the interface comprises a feature on the proximal shaft that allows the handle to move with respect to the proximal shaft when the removable shaft experiences forces greater than a threshold force.

In this or in combination with any other aspects of the disclosure, the interface comprises a set of features on the proximal shaft that allows the handle to move with respect to the proximal shaft when the removable shaft experiences forces greater than a threshold force.

In this or in combination with any other aspects of the disclosure, the interface comprises a feature or set of features that provides tactile feedback, audible feedback, visual feedback, or combinations thereof to a user when the removable shaft experiences forces greater than a threshold force.

In this or in combination with any other aspects of the disclosure, the distal tip section is configured to transmit force to the shaft and at least a portion of the forces experienced by the shaft are transmitted to the shaft by the distal tip section.

In an aspect of the disclosure, a kit for providing access to an anatomical target via a body lumen includes a handle coupled with a proximal shaft via an interface, wherein the interface is configured to provide tactile feedback, audible feedback, visual feedback, or combinations thereof to a user when the shaft experiences forces greater than a threshold force, and a set of removable shafts, each removable shaft configured to be coupled with the proximal shaft and configured to transmit force to the proximal shaft.

In this or in combination with any other aspects of the disclosure, the interface comprises a mechanical, electrical, magnetic, electromagnetic, and/or electro-mechanical feature that provides tactile feedback, audible feedback, visual feedback, or combinations thereof to a user when the shaft experiences forces greater than a threshold force.

In this or in combination with any other aspects of the disclosure, wherein the interface comprises a feature on the handle that is configured to prevent excess force on the body lumen when the shaft experiences forces greater than a threshold force.

In this or in combination with any other aspects of the disclosure, the interface comprises a set of features on the handle that are configured to prevent excess force on the body lumen when the shaft experiences forces greater than a threshold force.

In this or in combination with any other aspects of the disclosure, the interface comprises a feature on the shaft that is configured to prevent excess force on the body lumen when the shaft experiences forces greater than a threshold force.

In this or in combination with any other aspects of the disclosure, the interface comprises a set of features on the shaft that are configured to prevent excess force on the body lumen when the shaft experiences forces greater than a threshold force.

In this or in combination with any other aspects of the disclosure, the distal tip section is configured to transmit force to the shaft and at least a portion of the forces experienced by the shaft are transmitted to the shaft by the distal tip section.

In this or in combination with any other aspects of the disclosure, the set includes removable shafts of different sizes.

In this or in combination with any other aspects of the disclosure, the set includes removable shafts of different French scale sizes.

In this or in combination with any other aspects of the disclosure, the interface comprises a mechanical, electrical, magnetic, electromagnetic, and/or electro-mechanical feature that is configured to prevent excess force on the body lumen when the removable shaft experiences forces greater than a threshold force.

In this or in combination with any other aspects of the disclosure, the interface comprises a feature on the handle that is configured to prevent excess force on the body lumen when the removable shaft experiences forces greater than a threshold force.

In this or in combination with any other aspects of the disclosure, the interface comprises a set of features on the handle that is configured to prevent excess force on the body lumen when the removable shaft experiences forces greater than a threshold force.

In this or in combination with any other aspects of the disclosure, the interface comprises a feature on the proximal shaft that is configured to prevent excess force on the body lumen when the removable shaft experiences forces greater than a threshold force.

In this or in combination with any other aspects of the disclosure, the interface comprises a set of features on the proximal shaft that is configured to prevent excess force on the body lumen when the removable shaft experiences forces greater than a threshold force.

In this or in combination with any other aspects of the disclosure, the distal tip section is configured to transmit force to the shaft and at least a portion of the forces experienced by the shaft are transmitted to the shaft by the distal tip section.

In an aspect of the disclosure, a method of accessing an anatomical target via a body lumen includes advancing a force-limiting access system with the body lumen toward an anatomical target, wherein the force-limiting access system comprises a handle coupled with a shaft via an interface configured to maintain a positional relationship between the handle and the shaft while a distal tip section coupled with the shaft experiences forces less than a threshold force and to allow the handle to move with respect to the shaft when the distal tip section experiences forces greater than a threshold force, and noting any feedback related to movement of the handle with respect to the shaft, where the feedback is tactile, audible, visual, or combinations thereof.

In this or in combination with any other aspects of the disclosure, the method of claim includes waiting for the body lumen to relax after noting any feedback.

In this or in combination with any other aspects of the disclosure, the method includes resuming advancing the force-limiting access system with the body lumen toward an anatomical target.

In this or in combination with any other aspects of the disclosure, the method includes retracting the force-limiting access system and repeating the steps of the method using a new shaft having a different size.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude the inclusion of such modifications, variations, and/or additions to the present subject matter as would be readily apparent to one of ordinary skill in the art.

1.-21. (canceled)

22. A kit for providing access to an anatomical target via a body lumen, comprising:
a handle coupled with a proximal shaft via an interface, wherein the interface is configured to maintain the positional relationship between the handle and the proximal shaft while a force less than a threshold force is transmitted to the proximal shaft and to allow the handle to move with respect to the proximal shaft when a force greater than the threshold force is transmitted to the proximal shaft; and
a set of removable shafts, each removable shaft configured to be coupled with the proximal shaft and configured to transmit force to the proximal shaft.

23. The kit of claim 22, wherein the set includes removable shafts of different sizes or different French scale sizes.

24. The kit of claim 22, wherein the interface comprises a mechanical, electrical, magnetic, electromagnetic, and/or electro-mechanical feature that allows the handle to move with respect to the proximal shaft when the removable shaft experiences a force greater than the threshold force.

25. The kit of claim 22, wherein the interface comprises a feature or a set of features on the handle that allows the handle to move with respect to the proximal shaft when the removable shaft experiences a force greater than the threshold force.

26. The kit of claim 22, wherein the interface comprises a feature or a set of features on the proximal shaft that allows the handle to move with respect to the proximal shaft when the removable shaft experiences a force greater than the threshold force.

27. The kit of claim 22, wherein the interface comprises a projection or a set of projections on the handle and a detent or a set of detents on the proximal shaft.

28. The kit of claim 22, wherein the interface comprises a shaft coupling element, a handle coupling element, and a bistable element connecting the shaft coupling element to the handle coupling element, wherein the bistable element is configured to transition from a first stable configuration to a second stable configuration when the removable shaft experiences a force greater than the threshold force.

29. The kit of claim 22, wherein the interface comprises a shaft coupling element, a handle coupling element, and a buckling element connecting the shaft coupling element to the handle coupling element, wherein the buckling element is characterized by a stiffness that resists buckling until the removable shaft experiences a force greater than the threshold force.

30. The kit of claim 22, wherein the handle comprises an upper handle portion and a lower handle portion coupled to the proximal shaft, wherein the interface comprises an interlocking element positioned between the upper and lower handle portions.

31. The kit of claim 22, wherein the interface comprises a feature or set of features that provides tactile feedback, audible feedback, visual feedback, or combinations thereof to a user when the removable shaft experiences a force greater than the threshold force.

32. A kit for providing access to an anatomical target via a body lumen, comprising:
a handle coupled with a proximal shaft via an interface, wherein the interface is configured to provide tactile feedback, audible feedback, visual feedback, or combinations thereof to a user when the shaft experiences a force greater than a threshold force; and
a set of removable shafts, each removable shaft configured to be coupled with the proximal shaft and configured to transmit force to the proximal shaft, wherein the set includes removable shafts of different sizes or different French scale sizes.

33. The kit of claim 32, wherein the interface comprises a mechanical, electrical, magnetic, electromagnetic, and/or electro-mechanical feature that provides the tactile feedback, audible feedback, visual feedback, or combinations thereof to the user when the shaft experiences the force greater than the threshold force.

34. The kit of claim 32, wherein the interface comprises a feature or a set of features on the handle that is configured to prevent excess force on the body lumen when the proximal shaft experiences a force greater than the threshold force.

35. The kit of claim 32, wherein the interface comprises a feature or a set of features on the proximal shaft that is configured to prevent excess force on the body lumen when the removable shaft experiences a force greater than the threshold force.

36. (canceled)

37. The kit of claim 32, wherein the interface comprises a mechanical, electrical, magnetic, electromagnetic, and/or electro-mechanical feature that is configured to prevent excess force on the body lumen when the removable shaft experiences a force greater than the threshold force.

38. The kit of claim 32, wherein the interface comprises a feature or a set of features on the handle that is configured to prevent excess force on the body lumen when the removable shaft experiences a force greater than the threshold force; or
wherein the interface comprises a feature or set of features on the proximal shaft that is configured to prevent excess force on the body lumen when the removable shaft experiences a force greater than the threshold force.

39. (canceled)

40. The kit of claim 32, wherein the interface comprises a projection or a set of projections on the handle and a detent or a set of detents on the proximal shaft.

41. The kit of claim 32, wherein the interface comprises a shaft coupling element, a handle coupling element, and a bistable element connecting the shaft coupling element to the handle coupling element, wherein the bistable element is configured to transition from a first stable configuration to a second stable configuration when the removable shaft experiences a force greater than the threshold force.

42. The kit of claim 32, wherein the interface comprises a shaft coupling element, a handle coupling element, and a buckling element connecting the shaft coupling element to the handle coupling element, wherein the buckling element is characterized by a stiffness that resists buckling until the removable shaft experiences a force greater than the threshold force.

43. The kit of claim 32, wherein the handle comprises an upper handle portion and a lower handle portion coupled to the proximal shaft, wherein the interface comprises an interlocking element positioned between the upper and lower handle portions.

44.-48. (canceled)
